(19) United States
(12) Patent Application Publication
(10) Pub. No.: US 2023/0321441 A1
(43) Pub. Date: Oct. 12, 2023

(54) METHOD AND APPARATUS FOR NEUROMODULATION GUIDANCE USING SYMPTOM-WISE SENSORY PROFILE

(71) Applicant: Boston Scientific Neuromodulation Corporation, Valencia, CA (US)

(72) Inventors: Ismael Huertas Fernandez, Sevilla (ES); Que T. Doan, West Hills, CA (US)

(21) Appl. No.: 18/126,731

(22) Filed: Mar. 27, 2023

Related U.S. Application Data
(60) Provisional application No. 63/330,062, filed on Apr. 12, 2022.

(51) Int. Cl. A61N 1/36

(52) U.S. Cl. CPC A61N 1/36132

(57) ABSTRACT

An example of a system for delivering neurostimulation from a stimulation device to a patient may include a programming control circuit, a sensory profiling circuit, and a stimulation control circuit. The programming control circuit may be configured to program the stimulation device for controlling delivery of the neurostimulation according to one or more stimulation waveforms and one or more stimulation fields. The sensory profiling circuit may be configured to receive information regarding painful symptoms of the patient and to determine a pain sensory profile for the patient using the received information. The stimulation control circuit may be configured to determine a recommendation for one or more spinal cord stimulation (SCS) therapies using the determined pain sensory profile and to determine the one or more stimulation waveforms and the one or more stimulation fields using the recommended one or more SCS therapies.

### CLAIM OF PRIORITY

This application claims the benefit of U.S. Provisional Application No. 63/330,062, filed on Apr. 12, 2022, which is hereby incorporated by reference in its entirety.

### TECHNICAL FIELD

This document relates generally to neuromodulation and more particularly to a method and system for controlling delivery of neurostimulation to a patient using a symptom-wise sensory profile of the patient.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device is used to program the implantable neurostimulator with stimulation parameters controlling the delivery of the neurostimulation energy.

In one example, the neurostimulation energy is delivered to a patient in a form of electrical signals. The delivery is controlled using stimulation parameters that specify spatial (where to stimulate), temporal (when to stimulate), and informational (patterns of pulses directing the nervous system to respond as desired) aspects of a pattern of the electrical signals, Efficacy and efficiency of certain neurostimulation therapies can be improved, and their side-effects can be reduced, by customizing these stimulation parameters for a patient based on the patient's conditions and therapeutic objectives. While modern electronics can accommodate the need for generating sophisticated signal patterns, capability of a neurostimulation system depends on how stimulation parameters defining such a signal pattern can be determined and adjusted for the patient to ensure efficacy and efficiency of a therapy using neurostimulation when applied to the patient.

### SUMMARY

An Example (e.g., “Example 1”) of a system for delivering neurostimulation from a stimulation device to a patient is provided. The system may include a programming control circuit, a sensory profiling circuit, and a stimulation control circuit. The programming control circuit may be configured to program the stimulation device for controlling delivery of the neurostimulation according to one or more stimulation waveforms and one or more stimulation fields. The sensory profiling circuit may be configured to receive information regarding painful symptoms of the patient and to determine a pain sensory profile for the patient using the received information. The stimulation control circuit may be configured to determine a recommendation for one or more spinal cord stimulation (SCS) therapies using the determined pain sensory profile and to determine the one or more stimulation waveforms and the one or more stimulation fields using the recommended one or more SCS therapies.

In Example 2, the subject matter of Example 1 may optionally be configured to include a user interface including a presentation device, a user input device, and an interface control circuit including the sensory profiling circuit and the stimulation circuit, such that the sensory profiling circuit is configured to receive the information regarding painful symptoms of the patient using the user input device, and the stimulation control circuit is configured to present the recommendation using the presentation device.

In Example 3, the subject matter of Example 2 may optionally be configured such that the stimulation device includes an implantable stimulation device and optionally be configured to include an external programming device configured to be communicatively coupled to the implantable stimulation device and including the programming control circuit and the user interface.

In Example 4, the subject matter of Example 3 may optionally be configured to include a smartphone configured to include the external programming device.

In Example 5, the subject matter of any one or any combination of Examples 2 to 4 may optionally be configured such that the sensory profiling circuit is configured to present a symptom questionnaire using the presentation device, to receive answers to the symptom questionnaire using the user input device, and to produce the pain sensory profile using the received answers.

In Example 6, the subject matter of Example 5 may optionally be configured such that the symptom questionnaire includes a list of symptom types and a measure of severity for each symptom type of the list of symptom types.

In Example 7, the subject matter of Example 6 may optionally be configured such that the symptom questionnaire further includes a frequency for each symptom type of the list of symptom types. The frequency associates the each symptom type with each physical state of a list of physical states of the patient.

In Example 8, the subject matter of any one or any combination of Examples 2 to 7 may optionally be configured such that the sensory profiling circuit is configured to produce the pain sensory profile for a specified body area of the patient.

In Example 9, the subject matter of Example 8 may optionally be configured such that the sensory profiling circuit is configured to produce the pain sensory profile for each body area of multiple specified body areas of the patient to result in a pain sensory map for the patient, and the stimulation control circuit is configured to determine the recommendation for the one or more SCS therapies using the resulted pain sensory map.

In Example 10, the subject matter of Example 9 may optionally be configured such that the stimulation control circuit is configured to determine a sensory subtype based on the pain sensory map, and the stimulation control circuit is configured to determine the recommendation for the one or more SCS therapies using the determined sensory subtype.

In Example 11, the subject matter of Example 10 may optionally be configured such that the stimulation control circuit is configured to receive one or more additional factors and to determine the recommendation for the one or more SCS therapies using the determined sensory subtype and the received one or more additional factors. The one or more additional factors include at least one of a stimulation-related factor, a disease-related factor, a demographic factor, or a behavioral factor.

In Example 12, the subject matter of any one or any combination of Examples 10 and 11 may optionally be configured such that the stimulation control circuit is configured to determine the recommendation for the one or more SCS therapies using a predetermined relationship mapping known sensory subtypes to available SCS therapies.

In Example 13, the subject matter of any one or any combination of Examples 10 to 12 may optionally be configured such that the stimulation control circuit is further configured to determine a likelihood of suitability for the sensory subtype associated with each therapy of the one or more SCS therapies and to determine the one or more stimulation waveforms and the one or more stimulation fields based on the recommended one or more SCS therapies and the associated one or more likelihoods of suitability.

In Example 14, the subject matter of Example 13 may optionally be configured such that the stimulation control circuit is further configured to determine the likelihood of suitability for the sensory subtype associated with each therapy of the one or more SCS therapies using a predetermined lookup table providing the likelihood of suitability for each sensory subtype of the known sensory subtypes associated with each therapy of the available SCS therapies.

In Example 15, the subject matter of any one or any combination of Examples 13 and 14 may optionally be configured such that the stimulation control circuit is configured to present the recommendation showing the determined sensory subtype, one or more therapies selected from the available SCS therapies, and one or more likelihoods of suitability each associated with a therapy of the selected one or more therapies for the determined sensory subtype.

An example (e.g., “Example 16”) of a method for delivering neurostimulation from a stimulation device to a patient is also provided. The method may include receiving information regarding painful symptoms of the patient, determining a pain sensory profile for the patient using the received information using a processor of a programming device, determining a recommendation for one or more spinal cord stimulation (SCS) therapies using the determined pain sensory profile using the processor, determining one or more stimulation waveforms and one or more stimulation fields using the recommended one or more SCS therapies using the processor, and programming the stimulation device, using the programming device, for controlling delivery of the neurostimulation according to the one or more stimulation waveforms and the one or more stimulation fields.

In Example 17, the subject matter of receiving the information regarding painful symptoms of the patient as found in Example 16 may optionally include presenting a symptom questionnaire using a presentation device, receiving answers to the symptom questionnaire using a user input device, and producing the pain sensory profile using the received answers using the processor.

In Example 18, the subject matter of presenting the symptom questionnaire as found in Example 17 may optionally include presenting a list of symptom types, presenting measures of severity each associated with a symptom type of the list of symptom types, and presenting frequencies each associated with a symptom type of the list of symptom types and relating the each associated symptom type to one or more physical states of the patient.

In Example 19, the subject matter of determining the pain sensory profile for the patient as found in any one or any combination of Examples 16 to 18 may optionally include determining the pain sensory profile for a specified body area of the patient.

In Example 20, the subject matter of determining the pain sensory profile for the patient as found in Example 19 may optionally include determining the pain sensory profile for each body area of multiple specified body areas of the patient to result in a pain sensory map for the patient, and the subject matter of determining the recommendation for one or more SCS therapies using the determined pain sensory profile as found in Example 19 may optionally include determining the recommendation for one or more SCS therapies using the determined pain sensory map.

In Example 31, the subject matter of determining the recommendation for one or more SCS therapies using the determined pain sensory profile as found in Example 30 may optionally include determining a sensory subtype based on the pain sensory map and determining the recommendation for the one or more SCS therapies using the determined sensory subtype.

In Example 32, the subject matter of determining the sensory subtype as found in Example 31 may optionally include selecting the sensory subtype from known sensory subtypes, and the subject matter of determining the recommendation for the one or more SCS therapies as found in Example 31 may optionally include selecting the one or more SCS therapies from available SCS therapies using a predetermined relationship mapping the known sensory subtypes to the available SCS therapies.

In Example 33, the subject matter of determining the recommendation for the one or more SCS therapies using the determined sensory subtype as found in Example 32 may optionally further include determining a likelihood of suitability for the sensory subtype associated with each therapy of the one or more SCS therapies.

In Example 34, the subject matter of Example 33 may optionally further include presenting the recommendation for the one or more SCS therapies. The presentation includes presenting the determined sensory subtype, the one or more SCS therapies, and one or more likelihoods of suitability each associated with a therapy of the one or more SCS therapies.

An example (e.g., “Example 25”) of a non-transitory computer-readable storage medium is also provided. The non-transitory computer-readable storage medium includes instructions, which when executed by a system, cause the system to perform a method for delivering neurostimulation from a stimulation device to a patient. The method may include receiving information regarding painful symptoms of the patient, determining a pain sensory profile for the patient using the received information, determining a recommendation for one or more spinal cord stimulation (SCS) therapies using the determined pain sensory profile, determining one or more stimulation waveforms and one or more stimulation fields using the recommended one or more SCS therapies, and programming the stimulation device for controlling delivery of the neurostimulation according to one or more stimulation waveforms and one or more stimulation fields.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a neurostimulation system.

FIG. 2 illustrates an embodiment of a stimulation device and a lead system, such as may be implemented in the neurostimulation system of FIG. 1.

FIG. 3 illustrates an embodiment of a programming device, such as may be implemented in the neurostimulation system of FIG. 1.

FIG. 4 illustrates an embodiment of an implantable pulse generator (IPG) and an implantable lead system, such as an example implementation of the stimulation device and lead system of FIG. 2.

FIG. 5 illustrates an embodiment of an IPG and an implantable lead system, such as the IPG and lead system of FIG. 4, arranged to provide neurostimulation to a patient.

FIG. 6 illustrates an embodiment of portions of a neurostimulation system.

FIG. 7 illustrates an embodiment of an implantable stimulator and one or more leads of an implantable neurostimulation system, such as the implantable neurostimulation system of FIG. 6.

FIG. 8 illustrates an embodiment of an external programming device of an implantable neurostimulation system, such as the implantable neurostimulation system of FIG. 6.

FIG. 9 illustrates an embodiment of a system for controlling delivery of neurostimulation from a stimulation device, such as may be implemented in the neurostimulation system of FIG. 1 or the implantable neurostimulation system of FIG. 6.

FIG. 10 illustrates an embodiment of a user interface for controlling delivery of the neurostimulation in the system of FIG. 9.

FIGS. 11A and 11B illustrate an embodiment of a symptom area of a user interface, such as the user interface of FIG. 10. FIG. 11A shows an example of a symptom type being selected. FIG. 11B shows an example of another symptom type being selected.

FIG. 12 illustrates an embodiment of a sensory map area of a user interface, such as the user interface of FIG. 10.

FIG. 13 illustrates an embodiment of a lookup table for likelihood of suitability of an available therapy for a known sensory subtype.

FIG. 14 illustrates a specific example of the lookup table of FIG. 13.

FIG. 15 illustrates an alternative presentation of the lookup table of FIG. 14.

FIG. 16 illustrates an embodiment of a method for controlling delivery of neurostimulation from a stimulation device.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, a neurostimulation system that delivers neurostimulation to a patient and controls the delivery of the neurostimulation using the patient's symptom-wise sensory profile. In various embodiments, the neuromodulation system can include an implantable device configured to deliver neurostimulation (also referred to as neuromodulation) therapies, such as deep brain stimulation (DBS), spinal cord stimulation (SCS), peripheral nerve stimulation (PNS), and vagus nerve stimulation (VNS), and one or more external devices configured to program or adjust the implantable device for its operations and monitor the performance of the implantable device. Various operations of the implantable device, including sensing and stimulation, can be controlled using such one or more external devices.

In this document, unless noted otherwise, a “patient” includes a person receiving treatment delivered from, and/or monitored using, a neurostimulation system according to the present subject matter. A “user” includes a physician, other caregiver who examines and/or treats the patient using the neurostimulation system, or other person who participates in the examination and/or treatment of the patient using the neurostimulation system (e.g., a technically trained representative, a field clinical engineer, a clinical researcher, or a field specialist from the manufacturer of the neurostimulation system). A “pain sensory profile” includes a symptom-wise sensory profile that includes information related to painful symptoms of the patient and may not include etiologies and/or underlying medical conditions for the painful symptoms.

While neurostimulation, such as SCS, has been applied for pain management, there is a need for expanding indications (e.g., different types of pain, with different etiologies and symptoms) for such therapies. For each type of pain to be treatable by neurostimulation, stimulation parameters controlling the delivery of neurostimulation may need to be set and adjusted based on characteristics of that type of pain (e.g., location, severity, frequency, and type of sensation). For example, diabetic peripheral neuropathy (DPN), also known as painful diabetic peripheral neuropathy (pDPN), is a target indication for neurostimulation and is known to cause pain by a different mechanism than other pain etiologies due to its neurodegenerative process. A patient with DPN may suffer from a variety of sensory symptoms including numbness, allodynia, etc.

Neuropathic pain is characterized by manifestation of a wide variety of somatosensory symptoms. These include dysesthesia (burning, electric shock, stabbing, paresthesia, numbness, allodynia, etc.). While the tendency in medicine is to characterize patients by their etiology, patients with neuropathic pain of distinct chronic pain etiologies (DPN, chemotherapy-induced peripheral neuropathy (CIPN), HIV-induced neuropathy, central post-stroke pain (CPSP), etc.) may have similar somatosensory abnormalities or symptoms. Conversely, patients with the same etiology may experience different somatosensory symptoms. The present subject matter uses the findings that certain SCS therapies may be more effective for specific somatosensory profiles, and thus patients sharing the same or similar somatosensory profiles (as well as other factors), regardless of the etiology, may benefit from the same or similar SCS therapies. Neuropathic pain can be categorized based on a pain sensory profile using simple symptoms questionnaire administration. One example includes the neuropathic pain symptom inventory (NPSI) pain scale, which includes 10 pain descriptors each to be rated on a 0 (no symptom) to 10 (worst symptom imaginable) numeric rating scale. The 10 pain descriptors include: burning, electric shock, stabbing, pins and needles, tingling, squeezing, pressure, provoked by brushing, provoked by pressure, and provoked by cold. Different SCS paradigms may be suitable for different sensory profiles.

The present subject matter can identify neurostimulation therapies suitable for various pain sensory profiles. For example, for determining suitable SCS therapy treating chronic pain, a patient's pain sensory profile can be classified as one of subtypes of pain such as sensory loss, mechanical hypersensitivity (hyperalgesia and/or allodynia) or thermal hypersensitivity (hyperalgesia and/or allodynia) using an algorithm that relates the patient's pain sensory profile to one of multiple subtypes of pain. In various embodiments, the pain profile can be determined for multiple areas on the patient's body, thereby providing a pain sensory map. The pain sensory map can be used for the classification of the subtype of pain. In various embodiments, the multiple subtypes of pain are mapped to multiple sets of stimulation parameters and/or multiple stimulation programs. A set of stimulation parameters and/or a stimulation program can be selected as a recommendation for guiding the SCS therapy programming for the patient.

The present subject matter uses a patient's symptom-wise sensory profile, including a symptom map, to guide programming of a neurostimulation system for delivering SCS to manage pain with mono-symptom or multi-symptom etiologies. Sensory profile is a holistic quantification that can be used for a variety of indications. Examples of such indication include central post stroke pain (CPSP), post-traumatic peripheral pain (PTNP), human immunodeficiency virus (HIV)-induced pain, diabetic peripheral neuropathy (DPN), chemotherapy-induced peripheral neuropathy (CIPN), and small fiber neuropathy (SFN). It is noted that present subject matter recommends SCS programs and/or stimulation parameters based on the patient's painful symptom(s), rather than the etiology of pain. In other words, delivery of the SCS therapy is controlled using information indicating painful symptoms of the patient, rather than the underlying indications (causes or mechanisms of the pain).

Examples of symptom descriptors of chronic pain used for profiling the patient's pain symptoms for guiding SCS therapy programming include:
Neuropathic pain:
   numbness,
   dysesthesia (e.g., burning, cold, electric shock, and tingling), and
   hypersensitivity (e.g., tactile, mechanical, and thermal hypersensitivities), and
Nociceptive pain.
   These descriptors of neuropathic pain describe the pain without referring to the cause or mechanism. Hyperalgesia (increased sensitivity to pain or enhanced intensity of pain sensation) and allodynia (pain caused by a stimulus that does not normally cause pain) are two types of hypersensitivity to pain with different levels of severity (with allodynia being more severe). The nociceptive pain is a type of pain caused by damage to body tissue and may feel sharp, aching, or throbbing.

While SCS therapies and the pain sensory profile are specifically discussed as examples in this document, the present subject matter can be applied to other types of neurostimulation therapies and other types of sensory profiles. In various embodiments, the present subject matter determines a symptom-wise sensory profile based on the patient's symptoms and determines a neurostimulation therapy based on the symptom-wise sensory profile. The symptom-wise sensory profile can be related to a neurostimulation therapy defined by, for example, one or more stimulation programs and/or or one or more sets of stimulation parameters according to which the delivery of neurostimulation is controlled.

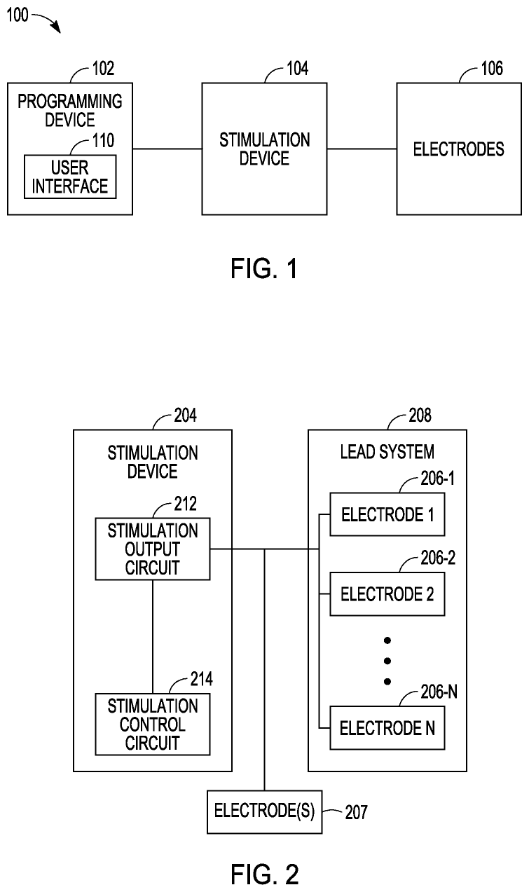

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets though electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are programmable by a user, such as a physician or other caregiver who treats the patient using system 100. Programming device 102 provides the user with accessibility to the user-programmable parameters. In various embodiments, programming device 102 is configured to be communicatively coupled to stimulation device via a wired or wireless link.

In various embodiments, programming device 102 can include a user interface 110 that allows the user to control the operation of system 100 and monitor the performance of system 100 as well as conditions of the patient including responses to the delivery of the neurostimulation. The user can control the operation of system 100 by setting and/or adjusting values of the user-programmable parameters.

In various embodiments, user interface 110 can include a graphical user interface (GUI) that allows the user to set and/or adjust the values of the user-programmable parameters by creating and/or editing graphical representations of various waveforms. Such waveforms may include, for example, a waveform representing a pattern of neurostimulation pulses to be delivered to the patient as well as individual waveforms that are used as building blocks of the pattern of neurostimulation pulses, such as the waveform of each pulse in the pattern of neurostimulation pulses. The GUI may also allow the user to set and/or adjust stimulation fields each defined by a set of electrodes through which one or more neurostimulation pulses represented by a waveform are delivered to the patient. The stimulation fields may each be further defined by the distribution of the current of each neurostimulation pulse in the waveform. In various embodiments, neurostimulation pulses for a stimulation period (such as the duration of a therapy session) may be delivered to multiple stimulation fields.

In various embodiments, system 100 can be configured for neurostimulation applications. User interface 110 can be configured to allow the user to control the operation of system 100 for neurostimulation. For example, system 100 as well as user interface 100 can be configured for SCS applications. Such SCS configuration includes various features that may simplify the task of the user in programming stimulation device 104 for delivering SCS to the patient, such as the features discussed in this document.

FIG. 2 illustrates an embodiment of a stimulation device 204 and a lead system 208, such as may be implemented in neurostimulation system 100. Stimulation device 204 represents an example of stimulation device 104 and includes a stimulation output circuit 212 and a stimulation control circuit 214. Stimulation output circuit 212 produces and delivers neurostimulation pulses. Stimulation control circuit 214 controls the delivery of the neurostimulation pulses from stimulation output circuit 212 using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses or each of collections of pulse intended to be delivered using the same combination of electrodes. In various embodiments, one or more additional electrodes 207 (each of which may be referred to as a reference electrode) can be electrically connected to stimulation device 204, such as one or more electrodes each being a portion of or otherwise incorporated onto a housing of stimulation device 204. Monopolar stimulation uses a monopolar electrode configuration with one or more electrodes selected from electrodes 206 and at least one electrode from electrode(s) 207. Bipolar stimulation uses a bipolar electrode configuration with two electrodes selected from electrodes 206 and none electrode(s) 207. Multipolar stimulation uses a multipolar electrode configuration with multiple (two or more) electrodes selected from electrodes 206 and none of electrode(s) 207.

In various embodiments, the number of leads and the number of electrodes on each lead depend on, for example, the distribution of target(s) of the neurostimulation and the need for controlling the distribution of electric field at each target. In one embodiment, lead system 208 includes 2 leads each having 8 electrodes.

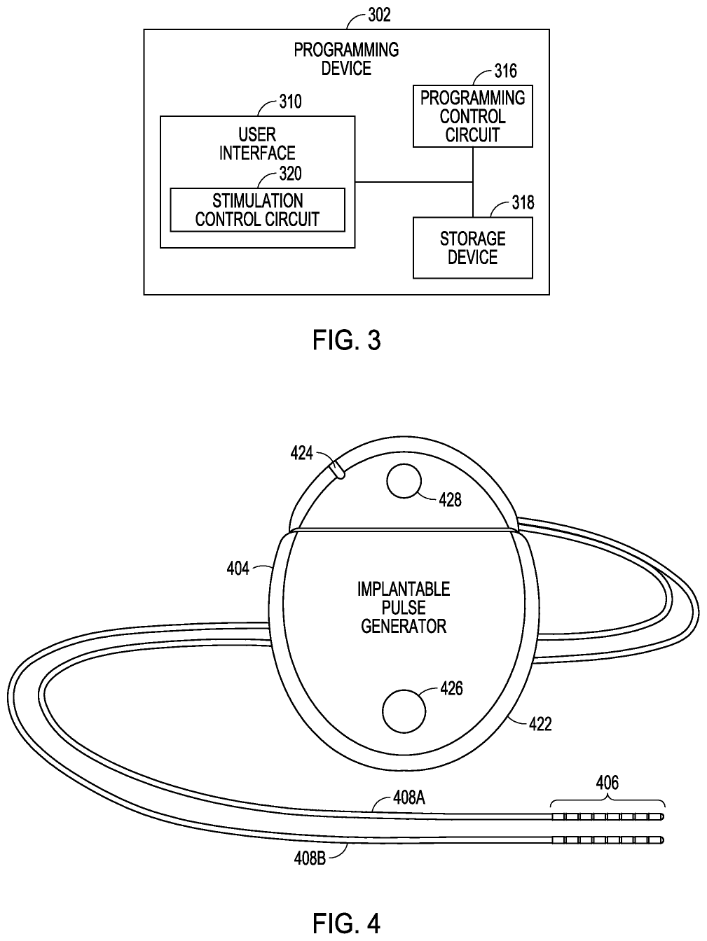

FIG. 3 illustrates an embodiment of a programming device 302, such as may be implemented in neurostimulation system 100. Programming device 302 represents an example of programming device 102 and includes a storage device 318, a programming control circuit 316, and a user interface 310. Programming control circuit 316 generates information representing or indicating the plurality of stimulation parameters that controls the delivery of the neurostimulation pulses according to a specified neurostimulation program that can define, for example, stimulation waveform and electrode configuration. User interface 310 represents an example of user interface 110 and includes a stimulation control circuit 320. Storage device 318 stores information used by programming control circuit 316 and stimulation control circuit 320, such as information about a stimulation device that relates the neurostimulation program to the plurality of stimulation parameters. In various embodiments, stimulation control circuit 320 can be configured to support one or more functions allowing for programming of stimulation devices, such as stimulation device 104 including its various embodiments as discussed in this document, according to one or more selected neurostimulation programs as discussed in this document.

In various embodiments, user interface 310 can allow for definition of a pattern of neurostimulation pulses for delivery during a neurostimulation therapy session by creating and/or adjusting one or more stimulation waveforms using a graphical method. The definition can also include definition of one or more stimulation fields each associated with one or more pulses in the pattern of neurostimulation pulses. As used in this document, a “neurostimulation program” can include the pattern of neurostimulation pulses including the one or more stimulation fields, or at least various aspects or parameters of the pattern of neurostimulation pulses including the one or more stimulation fields. In various embodiments, user interface 310 includes a GUI that allows the user to define the pattern of neurostimulation pulses and perform other functions using graphical methods. In this document, “neurostimulation programming” can include the definition of the one or more stimulation waveforms, including the definition of one or more stimulation fields.

In various embodiments, circuits of neurostimulation 100, including its various embodiments discussed in this document, may be implemented using a combination of hardware and software. For example, the circuit of user interface 110, stimulation control circuit 214, programming control circuit 316, and stimulation control circuit 320, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 4 illustrates an embodiment of an implantable pulse generator (IPG) 404 and an implantable lead system 408. IPG 404 represents an example implementation of stimulation device 204. Lead system 408 represents an example implementation of lead system 208. As illustrated in FIG. 4, IPG 404 that can be coupled to implantable leads 408A and 408B at a proximal end of each lead. The distal end of each lead includes electrical contacts or electrodes 406 for contacting a tissue site targeted for electrical neurostimulation. As illustrated in FIG. 1, leads 408A and 408B each include 8 electrodes 406 at the distal end. The number and arrangement of leads 408A and 408B and electrodes 406 as shown in FIG. 1 are only an example, and other numbers and arrangements are possible. In various embodiments, the electrodes are ring electrodes. The implantable leads and electrodes may be configured by shape and size to provide electrical neurostimulation energy to a neuronal target included in the subject's brain or configured to provide electrical neurostimulation energy to a nerve cell target included in the subject's spinal cord.

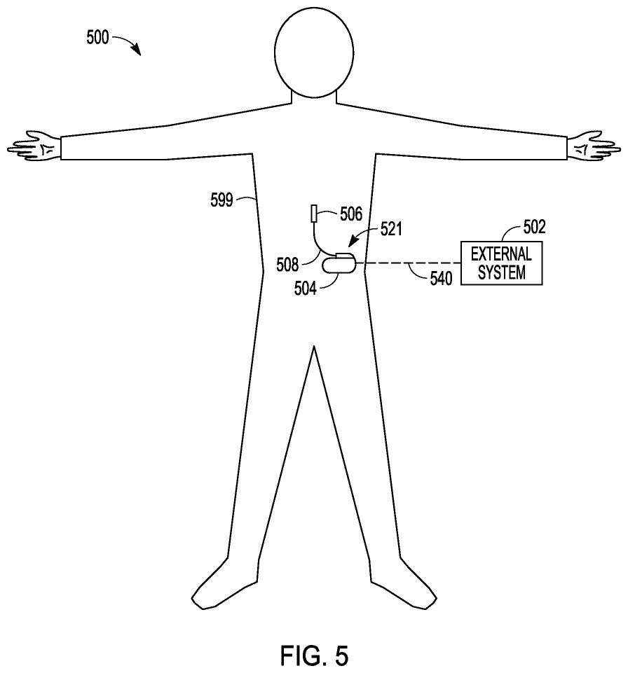

FIG. 5 illustrates an implantable neurostimulation system 500 and portions of an environment in which system 500 may be used. System 500 includes an implantable system 521, an external system 502, and a telemetry link 540 providing for wireless communication between implantable system 521 and external system 502. Implantable system 521 is illustrated in FIG. 5 as being implanted in the patient's body 599.

Implantable system 521 includes an implantable stimulator (also referred to as an implantable pulse generator, or IPG) 504, a lead system 508, and electrodes 506, which represent an example of stimulation device 204, lead system 208, and electrodes 206, respectively. External system 502 represents an example of programming device 302. In various embodiments, external system 502 includes one or more external (non-implantable) devices each allowing the user and/or the patient to communicate with implantable system 521. In some embodiments, external 502 includes a programming device intended for the user to initialize and adjust settings for implantable stimulator 504 and a remote control device intended for use by the patient. For example, the remote control device may allow the patient to turn implantable stimulator 504 on and off and/or adjust certain patient-programmable parameters of the plurality of stimulation parameters.

The sizes and sharps of the elements of implantable system 521 and their location in body 599 are illustrated by way of example and not by way of restriction. An implantable system is discussed as a specific application of the programming according to various embodiments of the present subject matter. In various embodiments, the present subject matter may be applied in programming any type of stimulation device that uses electrical pulses as stimuli, regarding less of stimulation targets in the patient's body and whether the stimulation device is implantable.

Returning to FIG. 4, the IPG 404 can include a hermetically-sealed IPG case 422 to house the electronic circuitry of IPG 404. IPG 404 can include an electrode 426 formed on IPG case 422. IPG 404 can include an IPG header 424 for coupling the proximal ends of leads 408A and 408B. IPG header 424 may optionally also include an electrode 428. Electrodes 426 and/or 428 represent embodiments of electrode(s) 207 and may each be referred to as a reference electrode.

Neurostimulation energy can be delivered in a monopolar (also referred to as unipolar) mode using electrode 426 or electrode 428 and one or more electrodes selected from electrodes 406. Neurostimulation energy can be delivered in a bipolar mode using a pair of electrodes of the same lead (lead 408A or lead 408B). Neurostimulation energy can be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 can include a control circuit that controls delivery of the neurostimulation energy. The control circuit can include a microprocessor, a digital signal processor, application specific integrated circuit (ASIC), or other type of processor, interpreting or executing instructions included in software or firmware. The neurostimulation energy can be delivered according to specified (e.g., programmed) modulation parameters. Examples of setting modulation parameters can include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

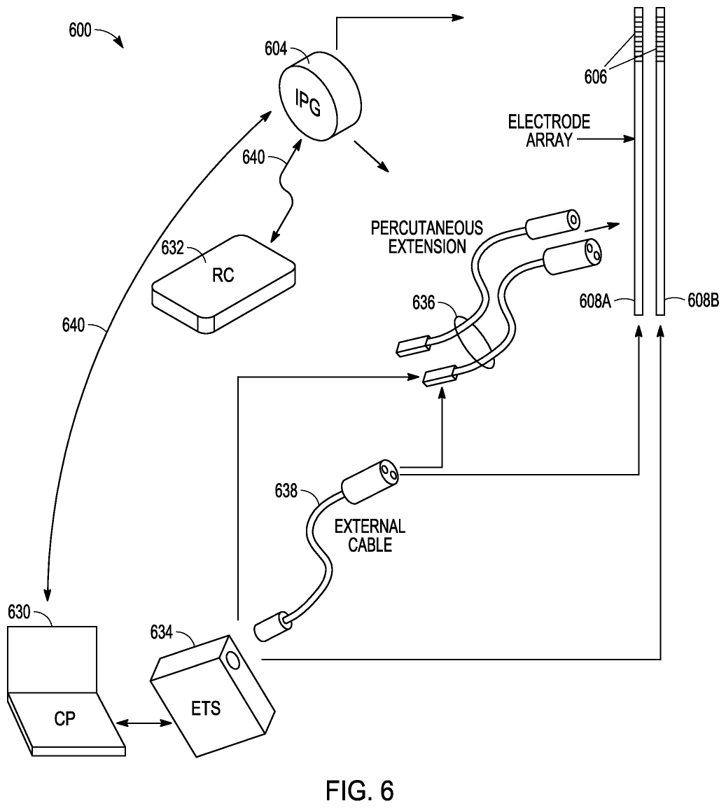

FIG. 6 illustrates an embodiment of portions of a neurostimulation system 600. System 600 includes an IPG 604, implantable neurostimulation leads 608A and 608B, an external remote controller (RC) 632, a clinician's programmer (CP) 630, and an external trial stimulator (EIS, also referred to as external trial modulator, ETM) 634. IPG 404 may be electrically coupled to leads 608A and 608B directly or through percutaneous extension leads 636. ETS 634 may be electrically connectable to leads 608A and 608B via one or both of percutaneous extension leads 636 and/or external cable 638. System 600 represents an example of system 100, with IPG 604 representing an embodiment of stimulation device 104, electrodes 606 of leads 608A and 608B representing electrodes 106, and CP 630, RC 632, and ETS 634 collectively representing programming device 102.

ETS 634 may be standalone or incorporated into CP 630. ETS 634 may have similar pulse generation circuitry as IPG 604 to deliver neurostimulation energy according to specified modulation parameters as discussed above. ETS 634 is an external device that is typically used as a preliminary stimulator after leads 408A and 408B have been implanted and used prior to stimulation with IPG 604 to test the patient's responsiveness to the stimulation that is to be provided by IPG 604. Because ETS 634 is external it may be more easily configurable than IPG 604.

CP 630 can configure the neurostimulation provided by ETS 634. If ETS 634 is not integrated into CP 630, CP 630 may communicate with ETS 634 using a wired connection (e.g., over a USB link) or by wireless telemetry using a wireless communications link 640. CP 630 also communicates with IPG 604 using a wireless communications link 640.

An example of wireless telemetry is based on inductive coupling between two closely-placed coils using the mutual inductance between these coils. This type of telemetry is referred to as inductive telemetry or near-field telemetry because the coils must typically be closely situated for obtaining inductively coupled communication. IPG 604 can include the first coil and a communication circuit. CP 630 can include or otherwise electrically connected to the second coil such as in the form of a wand that can be place near IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=κ/2π, where λ, is the wavelength of the transmitted electromagnetic energy. In one example, a communication range of an RF telemetry link is at least six feet but can be as long as allowed by the particular communication technology. RF antennas can be included, for example, in the header of IPG 604 and in the housing of CP 630, eliminating the need for a wand or other means of inductive coupling. An example is such an RF telemetry link is a Bluetooth® wireless link.

CP 630 can be used to set modulation parameters for the neurostimulation after IPG 604 has been implanted. This allows the neurostimulation to be tuned if the requirements for the neurostimulation change after implantation. CP 630 can also upload information from IPG 604.

RC 632 also communicates with IPG 604 using a wireless link 340. RC 632 may be a communication device used by the user or given to the patient. RC 632 may have reduced programming capability compared to CP 630. This allows the user or patient to alter the neurostimulation therapy but does not allow the patient full control over the therapy. For example, the patient may be able to increase the amplitude of neurostimulation pulses or change the time that a preprogrammed stimulation pulse train is applied. RC 632 may be programmed by CP 630. CP 630 may communicate with the RC 632 using a wired or wireless communications link. In some embodiments, CP 630 can program RC 632 when remotely located from RC 632.

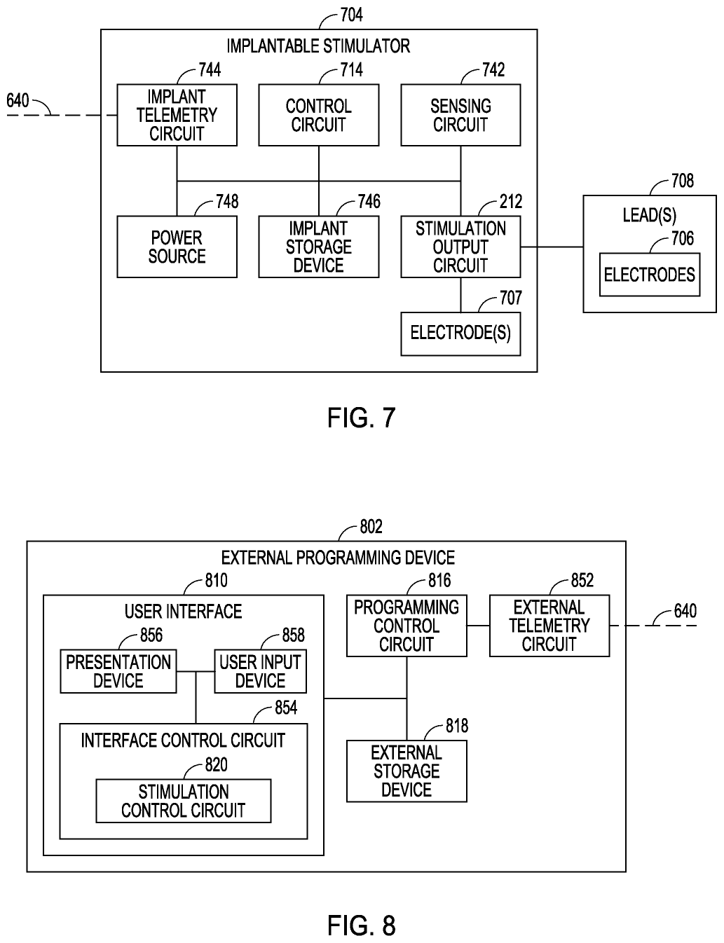

FIG. 7 illustrates an embodiment of implantable stimulator 704 and one or more leads 708 of an implantable neurostimulation system, such as implantable system 600. Implantable stimulator 704 represents an example of stimulation device 104 or 204 and may be implemented, for example, as IPG 604. Lead(s) 708 represents an example of lead system 208 and may be implemented, for example, as implantable leads 608A and 608B. Lead(s) 708 includes electrodes 706, which represents an example of electrodes 106 or 206 and may be implemented as electrodes 606.

Implantable stimulator 704 may include a sensing circuit 742 that provides the stimulator with a sensing capability, stimulation output circuit 212, a stimulation control circuit 714, an implant storage device 746, an implant telemetry circuit 744, a power source 748, and one or more electrodes 707. Sensing circuit 742 can one or more physiological signals for purposes of patient monitoring and/or feedback control of the neurostimulation. In various embodiments, sensing circuit 742 can sense one or more ESG signals using electrodes placed over or under the dura of the spinal cord, such as electrodes 706 (which can include epidural and/or intradural electrodes). In addition to one or more ESG signals, examples of the one or more physiological signals include neural and other signals each indicative of a condition of the patient that is treated by the neurostimulation and/or a response of the patient to the delivery of the neurostimulation. Stimulation output circuit 212 is electrically connected to electrodes 706 through one or more leads 708 as well as electrodes 707 and delivers each of the neurostimulation pulses through a set of electrodes selected from electrodes 706 and electrode(s) 707. Stimulation control circuit 714 represents an example of stimulation control circuit 214 and controls the delivery of the neurostimulation pulses using the plurality of stimulation parameters specifying the pattern of neurostimulation pulses. In one embodiment, stimulation control circuit 714 controls the delivery of the neurostimulation pulses using the one or more sensed physiological signals. Implant telemetry circuit 744 provides implantable stimulator 704 with wireless communication with another device such as CP 630 and RC 632, including receiving values of the plurality of stimulation parameters from the other device. Implant storage device 746 can store one or more neurostimulation programs and values of the plurality of stimulation parameters for each of the one or more neurostimulation programs. Power source 748 provides implantable stimulator 704 with energy for its operation. In one embodiment, power source 748 includes a battery. In one embodiment, power source 748 includes a rechargeable battery and a battery charging circuit for charging the rechargeable battery. Implant telemetry circuit 744 may also function as a power receiver that receives power transmitted from an external device through an inductive couple. Electrode(s) 707 allow for delivery of the neurostimulation pulses in the monopolar mode. Examples of electrode(s) 707 include electrode 426 and electrode 418 in IPG 404 as illustrated in FIG. 4.

In one embodiment, implantable stimulator 704 is used as a master database. A patient implanted with implantable stimulator 704 (such as may be implemented as IPG 604) may therefore carry patient information needed for his or her medical care when such information is otherwise unavailable. Implant storage device 746 is configured to store such patient information. For example, the patient may be given a new RC 632 and/or travel to a new clinic where a new CP 630 is used to communicate with the device implanted in him or her. The new RC 632 and/or CP 630 can communicate with implantable stimulator 704 to retrieve the patient information stored in implant storage device 746 through implant telemetry circuit 744 and wireless communication link 640 and allow for any necessary adjustment of the operation of implantable stimulator 704 based on the retrieved patient information. In various embodiments, the patient information to be stored in implant storage device 746 may include, for example, positions of lead(s) 708 and electrodes 706 relative to the patient's anatomy (transformation for fusing computerized tomogram (CT) of post-operative lead placement to magnetic resonance imaging (MRI) of the brain), clinical effect map data, objective measurements using quantitative assessments of symptoms (for example using micro-electrode recording, accelerometers, and/or other sensors), and/or any other information considered important or useful for providing adequate care for the patient. In various embodiments, the patient information to be stored in implant storage device 746 may include data transmitted to implantable stimulator 704 for storage as part of the patient information and data acquired by implantable stimulator 704, such as by using sensing circuit 742.

In various embodiments, sensing circuit 742 (if included), stimulation output circuit 212, stimulation control circuit 714, implant telemetry circuit 744, implant storage device 746, and power source 748 are encapsulated in a hermetically sealed implantable housing or case, and electrode(s) 707 are formed or otherwise incorporated onto the case. In various embodiments, lead(s) 708 are implanted such that electrodes 706 are placed on and/or around one or more targets to which the neurostimulation pulses are to be delivered, while implantable stimulator 704 is subcutaneously implanted and connected to lead(s) 708 at the time of implantation.

FIG. 8 illustrates an embodiment of an external programming device 802 of an implantable neurostimulation system, such as system 600. External programming device 802 represents an example of programming device 102 or 302, and may be implemented, for example, as CP 630 and/or RC 632. External programming device 802 includes an external telemetry circuit 852, an external storage device 818, a programming control circuit 816, and a user interface 810.

External telemetry circuit 852 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one embodiment, external telemetry circuit 852 also transmits power to implantable stimulator 704 through an inductive couple.

In various embodiments, wireless communication link 640 can include an inductive telemetry link (near-field telemetry link) and/or a far-field telemetry link (RF telemetry link). This can allow for patient mobility during programming and assessment when needed. For example, wireless communication link 640 can include at least a far-field telemetry link that allows for communications between external programming device 802 and implantable stimulator 704 over a relative long distance, such as up to about 20 meters. External telemetry circuit 852 and implant telemetry circuit 744 each include an antenna and RF circuitry configured to support such wireless telemetry.

External storage device 818 stores one or more stimulation waveforms for delivery during a neurostimulation therapy session, such as a SCS therapy session, as well as various parameters and building blocks for defining one or more waveforms. The one or more stimulation waveforms may each be associated with one or more stimulation fields and represent a pattern of neurostimulation pulses to be delivered to the one or more stimulation field during the neurostimulation therapy session. In various embodiments, each of the one or more stimulation waveforms can be selected for modification by the user and/or for use in programming a stimulation device such as implantable stimulator 704 to deliver a therapy. In various embodiments, each waveform in the one or more stimulation waveforms is definable on a pulse-by-pulse basis, and external storage device 818 may include a pulse library that stores one or more individually definable pulse waveforms each defining a pulse type of one or more pulse types. External storage device 818 also stores one or more individually definable stimulation fields. Each waveform in the one or more stimulation waveforms is associated with at least one field of the one or more individually definable stimulation fields. Each field of the one or more individually definable stimulation fields is defined by a set of electrodes through a neurostimulation pulse is delivered. In various embodiments, each field of the one or more individually definable fields is defined by the set of electrodes through which the neurostimulation pulse is delivered and a current distribution of the neurostimulation pulse over the set of electrodes. In one embodiment, the current distribution is defined by assigning a fraction of an overall pulse amplitude to each electrode of the set of electrodes. Such definition of the current distribution may be referred to as “fractionalization” in this document. In another embodiment, the current distribution is defined by assigning an amplitude value to each electrode of the set of electrodes. For example, the set of electrodes may include 2 electrodes used as the anode and an electrode as the cathode for delivering a neurostimulation pulse having a pulse amplitude of 4 mA. The current distribution over the 2 electrodes used as the anode needs to be defined. In one embodiment, a percentage of the pulse amplitude is assigned to each of the 2 electrodes, such as 75% assigned to electrode 1 and 25% to electrode 2. In another embodiment, an amplitude value is assigned to each of the 2 electrodes, such as 3 mA assigned to electrode 1 and 1 mA to electrode 2. Control of the current in terms of percentages allows precise and consistent distribution of the current between electrodes even as the pulse amplitude is adjusted. It is suited for thinking about the problem as steering a stimulation locus, and stimulation changes on multiple contacts simultaneously to move the locus while holding the stimulation amount constant. Control and displaying the total current through each electrode in terms of absolute values (e.g., mA) allows precise dosing of current through each specific electrode. It is suited for changing the current one contact at a time (and allows the user to do so) to shape the stimulation like a piece of clay (pushing/pulling one spot at a time).

Programming control circuit 816 represents an example of programming control circuit 316 and generates the information representing or indicating the plurality of stimulation parameters, which is to be transmitted to implantable stimulator 704. For example, programming control circuit 816 can generate the information based on a specified neurostimulation program (e.g., the pattern of neurostimulation pulses as represented by one or more stimulation waveforms and one or more stimulation fields, or at least certain aspects of the pattern). The neurostimulation program may be created and/or adjusted by the user using user interface 810 and stored in external storage device 818. In various embodiments, programming control circuit 816 can check values of the plurality of stimulation parameters against safety rules to limit these values within constraints of the safety rules. In one embodiment, the safety rules are heuristic rules.

User interface 810 represents an example of user interface 310 and allows the user to define the pattern of neurostimulation pulses and perform various other monitoring and programming tasks. User interface 810 includes a display screen 856, a user input device 858, and an interface control circuit 854. Display screen 856 may include any type of interactive or non-interactive screens, and user input device 858 may include any type of user input devices that supports the various functions discussed in this document, such as touchscreen, keyboard, keypad, touchpad, trackball, joystick, and mouse. In one embodiment, user interface 810 includes a GUI. The GUI may also allow the user to perform any functions discussed in this document where graphical presentation and/or editing are suitable as may be appreciated by those skilled in the art.

Interface control circuit 854 controls the operation of user interface 810 including responding to various inputs received by user input device 858 and defining the one or more stimulation waveforms. Interface control circuit 854 includes a stimulation control circuit 820.

In various embodiments, external programming device 802 can have operation modes including a composition mode and a real-time programming mode. Under the composition mode (also known as the pulse pattern composition mode), user interface 810 is activated, while programming control circuit 816 is inactivated. Programming control circuit 816 does not dynamically updates values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms and transmits the plurality of stimulation parameters with the updated values to implantable stimulator 704.

Stimulation control circuit 820 represents an example of stimulation control circuit 320 and can be configured to determine information related to a therapy based on which programming control circuit 816 can generate the information for programming implantable stimulator 704. Such information related to the therapy can include a neurostimulation program (e.g., the pattern of neurostimulation pulses) or parameters controlling the delivery of the neurostimulation (e.g., the one or more stimulation waveforms and one or more stimulation fields defining the pattern of neurostimulation pulses). In various embodiments, stimulation control circuit 820 can determine one or more SCS therapies based on one or more symptom-wise sensory profiles of information derived from the one or more symptom-wise sensory profiles. In various embodiments, stimulation control circuit 820 can generate a recommendation for the one or more SCS therapies and present the recommendation using presentation device 856 for the user (e.g., using CP 630) and/or the patient (e.g., using RC 632) to set or adjust the settings of implantable stimulator 704.

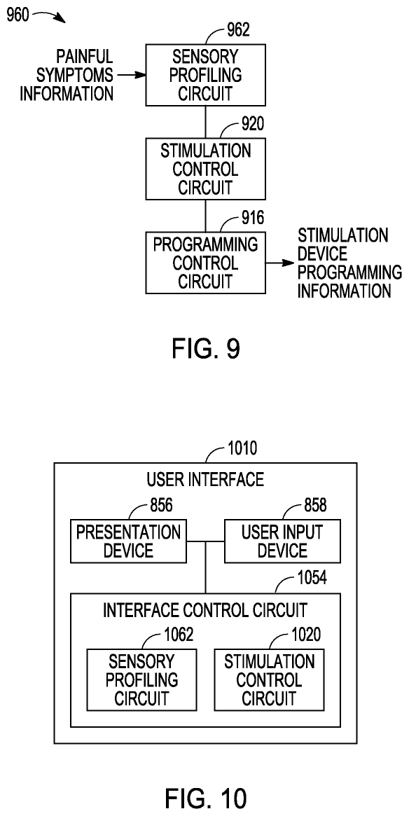

FIG. 9 illustrates an embodiment of a system 960 for controlling delivery of neurostimulation from a stimulation device, such as any stimulation device discussed in this document (e.g., stimulation device 104, stimulation device 204, IPG 404, implantable stimulator 504, IPG 604, or implantable stimulator 704). In various embodiments, system 960 can be implemented in a neurostimulation system, such as any neurostimulation system discussed in this document (e.g., system 100, 500, or 600). For example, the illustrated components of system 960 can be implemented in a programming device for the stimulation device, such as any programming device discussed in this document (e.g., programming device 102, programming device 302, external system 502, CP 630, RC 632, or external programming device 802).

In the illustrated embodiment, system 960 includes a sensory profiling circuit 962, a stimulation control circuit 920, and a programming control circuit 916. Sensory profiling circuit 962 can receive information regarding painful symptoms of the patient and determine a pain sensory profile for the patient using the received painful symptoms information. Stimulation control circuit 920 can represent an example of stimulation control circuit 320 or 820 and can determine a recommendation for one or more neurostimulation therapies (e.g., one or more SCS) therapies) using the pain sensory profile determined by sensory profiling circuit 962 and can determine one or more stimulation waveforms and one or more stimulation fields for the recommended one or more neuromodulation therapies. Programming control circuit 916 can represent an example of programming control circuit 316 or 816 and can produce stimulation device programming information for programming to stimulation device to control the delivery of neurostimulation according to the one or more stimulation waveforms and one or more stimulation fields determined by stimulation control circuit 920. In various embodiments, the one or more stimulation waveforms each define a temporal pattern of energy of the neurostimulation (e.g., a pattern of neurostimulation pulses, and the one or more stimulation fields each define a spatial distribution of the energy of the neurostimulation (e.g., over electrodes selected from a plurality of electrodes coupled to the stimulation device).

FIG. 10 illustrates an embodiment of a user interface 1010 for controlling delivery of the neurostimulation in the system of FIG. 9. User interface 1010 can in implemented in a neurostimulation system, such as any neurostimulation system discussed in this document (e.g., system 100, 500, or 600). For example, user interface 1010 can be implemented user interface 110. 310, or 810. User interface 101 can include presentation device 856, user input device 858, and an interface control circuit 1054. Interface control circuit 1054 can represent an example of interface control circuit 854 and includes a sensory profiling circuit 1062 and a stimulation control circuit 1020.

Sensory profiling circuit 1062 can represent an example of sensory profiling circuit 962 and can receive information regarding painful symptoms of the patient and determine a pain sensory profile for the patient using the received information. In one embodiment, sensory profiling circuit 1062 receives the information regarding painful symptoms of the patient by represent a symptom questionnaire using presentation device 856 and receiving answers to the symptom questionnaire using user input device 858. The answers can be entered by the user (e.g., using CP 630 including user interface 1010) or by the patient (e.g., using RC 632 including user interface 1010). In one embodiment, RC 632 is implemented in a mobile device such as a smartphone, and the patient is allowed to use RC 632 for self-reporting painful symptoms for adjusting the settings of his/her stimulation device.

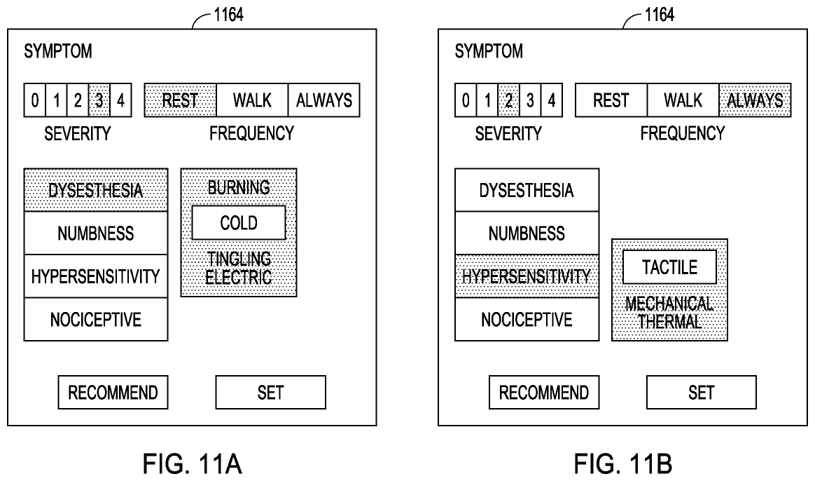

FIGS. 11A and 11B illustrate an embodiment of a symptom area 1164 of a user interface, such as user interface 1010. Symptom area 1164 can be presented using presentation device 856, such as displayed on a screen. In the illustrated embodiment, the symptom questionnaire is displayed in symptom area 1164 and includes a menu of symptom types, a severity scale, and a frequency. The menu of symptom types (e.g., including pain descriptors) includes dysesthesia (with a submenu including burning, cold, tingling, and electric [shock]), numbness, hypersensitivity (with a submenu including tactile, mechanical, and thermal), and nociceptive. User input device 868 is used to select one or more symptom types perceived by the patient. If a symptom type with a submenu is selected, the submenu will be displayed for selection. For each symptom type selected, user input device 868 is used to selected a number one the severity scale. In the illustrated example, the severity scale include 0 (no symptom) to 4 (worst). In various embodiments, the severity scale can include any numerical range (e.g., 0-5 or 0-10, depending on the desired resolution) and/or other symbols (e.g., the Wong-Baker Faces). For each symptom type selected, user input device 868 is also used to select a frequency, which indicates a variety of applicable activities or physical states of the patient when pain is perceived. In the illustrated example, the frequency includes rest (pain felt at rest), walk (pain felt during walking), and always (pain felt constantly). Additional examples of frequency include daytime, nighttime, exercise, sitting, standing, coughing, and stretching.

FIG. 11A shows an example of dysesthesia being selected as the symptom type. Upon selection of dysesthesia, the submenu (burning, cold, tingling, and electric [shock]) is displayed, and the illustrated example shows cold is selected. The illustrated example further shows that for dysesthesia/cold, severity of 3 (out of 4) is selected, and rest is selected as the frequency of when dysesthesia/cold is felt. FIG. 11B shows an example of hypersensitivity being selected as the symptom type. Upon selection of hypersensitivity, the submenu (b tactile, mechanical, and thermal) is displayed, and the illustrated example shows tactile is selected. The illustrated example further shows that for tactile hypersensitivity, severity of 2 (out of 4) is selected, and rest is selected as the frequency (i.e., constant tactile hypersensitivity).

After completion of the symptoms questionnaire, sensory profiling circuit 1062 can produce a pain sensory profile using the answers to the symptom questionnaire. The pain sensory profile can include all the information obtained by completing the symptom questionnaire with answers specific to the patient. In various embodiments, sensory profiling circuit 1062 can produce a pain sensory profile for a specified body area of the patient using a questionnaire for receiving answers specific to that specified body area.

The symptom questionnaire as shown in FIGS. 11A and 11B is an example discussed herein for the purpose of illustration rather than restriction. In various embodiments, the symptom questionnaire can include any pain descriptors as the symptom types. An example of descriptors for neuropathic pain include those in the NPSI: (1) burning, (2) electric shock, (3) stabbing, (4) pins & needles, (5) tingling, (6) squeezing, (7) pressure, (8) provoked by brushing, (9) provoked by pressure, and (10) provoked by cold. Another example of descriptors for neuropathic pain include those in the QST: (1) sensory thresholds, (2) static mechanical allodynia, (3) dynamic mechanical allodynia, (4) punctate hyperalgesia, (5) temporal summation, (6) cold allodynia, and (7) cold hyperalgesia. Freeman et al., “Sensory profile of patients with neuropathic pain based on the neuropathic pain symptoms and signs”, Pain, 155: 367-376, 2014. The symptom types included in the symptom questionnaire can also include symptoms of lost or diminished sensation. In various embodiments, the symptom types included in the symptom questionnaire can include negative (lost or diminished sensation) and positive (painful) sensory symptoms. An example of descriptors of negative and positive sensory symptoms includes:
negative symptoms (e.g., hypoaesthesia, pall-hypoaesthesia, hypoalgesia, and thermal hypoaesthesia);
spontaneous sensations or pain (e.g., paresthesia, paroxysmal pain, and superficial pain); and
evoked pain (e.g., mechanical dynamic allodynia, mechanical static allodynia, mechanical punctate, pin-prick hyperalgesia, temporal summation, cold hyperalgesia, heat hyperalgesia, and mechanical deep somatic hyperalgesia).
   Baron et al., “Neuropathic pain: diagnosis, pathophysiological mechanisms, and treatment”, www.the lancet.com/neurology, vol. 9, August, 2010. In various embodiments, the symptom types included in the symptom questionnaire can include those selected from the various descriptors including, but not being limited to, the descriptors of neuropathic pain and the descriptors of negative and positive sensory symptoms listed herein. For example, for the purpose of determining one or more SCS therapies, the symptom types included in the symptom questionnaire can include all the pain descriptors that are, or associated with, indications of available SCS therapies.

Studies have shown that clusters representing patients having distinct pain sensory profiles can be identified, that such clusters may not exclusively relate to the etiology of neuropathic pain, and an approach to determining a pain therapy based on such symptom-based clusters, rather than etiology, may improve a patient's response to the pain therapy. Distinct neuropathic pain sensory profiles with large variability were found within each etiology, which suggests distinct pathophysiologic mechanisms and hence, different pain treatment therapies or therapy settings. The present subject matter determines one or more therapies and/or therapy settings for a patient based on the pain sensory profile of the patient. For example, the patient can be identified with a sensory subtype based on pain sensory profile, as further discussed below. The sensory subtype can be selected, for example, from sensory subtypes corresponding to the clusters representing patients having distinct pain sensory profiles. In other words, the patient can be identified with a subgroup of patients having similar pain sensory profiles (e.g., pain sensory profiles characterizing painful symptoms with similar symptom types, scales, and/or frequencies). The one or more therapies can then be determined (e.g., for a recommendation) using the sensory subtype or pain sensory profile-based patient subgroup, rather than using or basing on the pain etiology. Once a relationship between the one or more therapies and the sensory subtype (or pain sensory profile-based patient subgroup) is determined (e.g., empirically), the present subject matter can be applied to program a stimulation device (e.g., stimulation device 102 including its various embodiments discussed in this document) based on the symptom-wise sensory profile (e.g., determined using user interface 1010).

In various embodiments, together with the pain sensory profile, other factors or variables can be included in the relationship between the one or more therapies and the sensory subtype, to provide for more accurate prediction of possible outcomes and hence a better recommendation. These factors can be stimulation-related (e.g., paresthesia-pain coverage or map, SCS-elicited paresthesia sensation map, perception-threshold, electrical field configuration, and the like), etiology-related (e.g., disease duration, baseline pain intensity, and the like), demographical (e.g., the patient's age, gender, and the like), and/or behavioral (e.g., the patient's levels of activity, mood, personality traits, and the like).

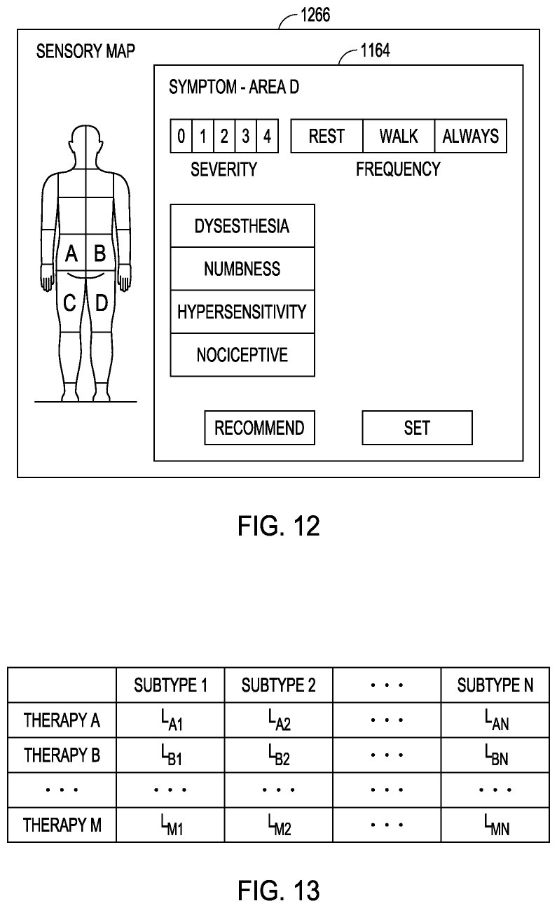

FIG. 12 illustrates an embodiment of a sensory map area 1266 of a user interface, such as user interface 1010. Sensory map area 1266 can be presented using presentation device 856, such as displayed on the screen. Sensory profiling circuit 1062 can produce a pain sensory profile for each body area of multiple specified body areas to result in a pain sensory map for the patient. In the illustrated embodiment, a representation of the body of the patient is displayed with the multiple specified body areas marked (e.g., body areas A-D, as shown). System area 1164 repeats process of presenting the symptom questionnaire and receiving the answers, as discussed above with reference to FIGS. 11A and 11B, for each of body areas A-D, one at a time. The pain sensory map includes the pain sensory profiles determined for all of the body areas A-D. In various embodiments, body areas to be included in the pain sensory map can be specified based on the patient's input, known or suspected indications, and other factors relating the patient's symptoms to candidate neurostimulation therapies.

In the illustrated embodiment, symptom area 1164 includes a “RECOMMEND” button and a “SET” button, which can be hit using user input device to produce a recommendation command and a set command, respectively. In response to the recommendation command, stimulation control circuit 1020 produces a recommendation for one or more neurostimulation therapies, as further discussed below. In response to the set command, sensory profiling circuit 1062 produces and saves a pain sensory profile with the answers to the symptom questionnaire that are already received (e.g., for a specified body area).

Symptom area 1164 and sensory map 1266 are shown in FIGS. 11A. 11B, and 12 by way of example, and not by way of restriction, to illustrate a manner by which the patient's pain sensory profile and pain sensory map can be obtained. In various embodiments, the patient's pain sensory profile(s) and/or pain sensory map(s) can be obtained in various ways and with various specific contents, depending on, for example, the algorithm that relates the patient's pain sensory profile(s) and/or pain sensory map(s) to neurostimulation therapies and design considerations and preferences for the user interface, as understood by those skilled in the art.

Stimulation control circuit 1020 can represent an example of stimulation control circuit 920 and can determine a recommendation for one or more neurostimulation therapies (e.g., one or more SCS therapies) using the pain sensory profile determined by sensory profiling circuit 1062 and can determine one or more stimulation waveforms and one or more stimulation fields for the recommended one or more neuromodulation therapies. In various embodiments, the one or more stimulation fields can be defined by targeted anatomical regions (e.g., spinal cord levels) and/or active electrodes, with fractionalization if applicable, when the electrodes are placed over the patient's spinal cord. The one or more stimulation waveforms can define a pattern of neurostimulation pulses.

In various embodiments, stimulation control circuit 1020 can determine a sensory subtype for the patient based on the pain sensory profile of the patient and then determine the recommendation for the one or more neurostimulation therapies using the sensory subtype. In various embodiments, stimulation control circuit 1020 can determine the sensory subtype by selecting the sensory subtype from multiple predetermined sensory subtypes. In various embodiments in which a sensory map is determined for the patient with multiple specified body areas, stimulation control circuit 1020 can determine the sensory subtype based on the pain sensory map. For example, stimulation control circuit 1020 can execute an algorithm that receives the pain sensory profile for each body area and determines a sensory subtype using the pain sensory profile based on known definition of sensory subtypes in terms of painful symptoms (e.g., the symptom types and their severities and frequencies).

For the purpose of illustration, rather than restriction, an example of the sensory subtypes for the purpose of determining SCS therapies, as discussed in this document, includes sensory loss, mechanical hypersensitivity (including hyperalgesia and/or allodynia), and thermal hypersensitivity (including hyperalgesia and/or allodynia). In one embodiment, the algorithm that receives the pain sensory profile for each body area and determines a sensory subtype using the pain sensory profile includes a cluster analysis that clusters pain sensory profiles sharing the same or similar characteristic painful symptoms (e.g., in terms of the symptom types, pain scales, and frequencies) into the sensory subtypes. In various embodiments, the sensory subtypes for the purpose of determining the SCS therapies can include all the sensory subtypes for which one or more SCS therapies are available. In various other embodiments, the sensory subtypes for the purpose of determining the SCS therapies can include all the sensory subtypes for which one or more SCS therapies are available as well as all the sensory subtypes for which other therapies can be recommended.

In various embodiments, stimulation control circuit 1020 determines one or more SCS therapies using the sensory subtype determined for the patient. The one or more SCS therapies can each include the one or more stimulation waveforms and the one or more stimulation fields. For example, the one or more SCS therapies can each include one or more neurostimulation programs each defining the one or more stimulation waveforms and the one or more stimulation fields over an SCS therapy session. Stimulation control circuit 1020 can also determine a likelihood of suitability for the sensory subtype associated with each therapy of the one or more SCS therapies using a relationship between known sensory subtypes and available SCS therapies. This relationship can be empirically established with a population of patients suffering from all of the known sensory subtypes and treated with the available SCS therapies with various levels of efficacy. In various embodiments, such a relationship can be established as a lookup table or illustrated as the lookup table (though presented in another format).

FIGS. 13-15 illustrate examples of such lookup tables. FIG. 13 illustrates an embodiment of a lookup table for likelihood of suitability of an available therapy for a known sensory subtype. The look-up table show Therapy i (i=1, 2, . . . M), Subtype j (j=1, 2, . . . N), and Lᵢⱼ, which is the Likelihood of suitability for Subtype i associated with Therapy j. In one embodiment, the sum for likelihoods of suitability for all the Therapies A-M for each Subtype being 1 (100%), and the likelihoods of suitability for each therapy is a fraction of 1 (a percentage less than 100%). That is, L_{A1}+L_{B1}+ . . . +L_{M1}=1 or 100%; L_{A2}+L_{B2}+ . . . +L_{M21}=1 or 100%; . . . L_{AN}+L_{BN}+ . . . +L_{MN}=1 or 100%. The lookup table can be generated empirically to include all available therapies and all known subtypes. In an example of SCS, the sensory subtypes include:
Subtype 1: Sensory loss;
Subtype 2: Mechanical allodynia; and
Subtype 3: Thermal allodynia.

The available SCS therapies (e.g., stimulation programs) include:
Therapy A: “Paresthesia-Based”, a low frequency (e.g., 40-100 Hz) paresthesia-based therapy;
Therapy B: “Sub-P High Rate”, a high frequency (e.g., 200-1,200 Hz) sub-perception therapy;
Therapy C: “Sub-P”, a low frequency (e.g., 90 Hz) active recharge sub-perception therapy;
Therapy D: “Combination”, a combination of low frequency paresthesia-based therapy and higher frequency sub-perception therapy; and
Therapy F: “Other”, other type of SCS therapeutic option based on variation of one or more of stimulation settings (e.g., stimulation field and/or stimulation waveform settings).
   This example, with 3 sensory subtypes and 5 available SCS therapies, is discussed herein for the purpose of illustration rather than restriction. In various embodiments, the present subject matter can be applied to determine one or more therapies (e.g., for recommending to the user and/or the patient) from any available therapies each with indication established in terms of sensory subtypes. The available therapies can include SCS and other neurostimulation therapies as well as other types of pain control therapies. The sensory subtypes can result from clusters of symptom characteristics extracted from the pain sensory profiles.

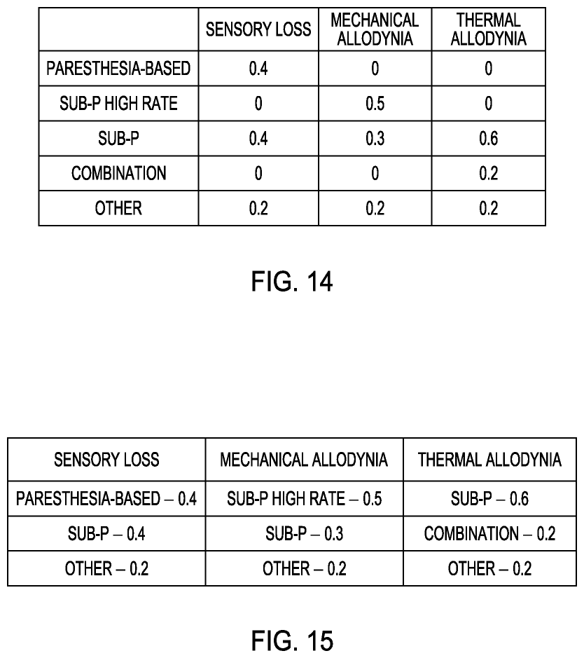

FIG. 14 illustrates a specific example of the lookup table of FIG. 13 with the above Subtypes 1-3 and Therapies A-F. FIG. 15 illustrates an alternative presentation of the lookup table of FIG. 14. In various embodiments, the lookup table can be in any operable format.

Stimulation control circuit 1020 can generate the recommendation for one or more SCS therapies based on the sensory subtype determined by sensory profiling circuit 1062 and the relationship between the known sensory subtypes and the available SCS therapies (e.g., the lookup table as illustrated in FIGS. 11-13). Once the recommendation for the one or more SCS therapies is accepted by the user or the patient, stimulation control circuit 1020 can determine the one or more stimulation waveforms and the one or more stimulation fields based on the accepted one or more SCS therapies. In various embodiments, additional factors, such as the patient's perception threshold and pain-paresthesia overlap, can be included in the generation of the recommendation and/or the determination of the one or more stimulation waveforms and the one or more stimulation fields. In various embodiments, stimulation control circuit 1020 can present the recommendation using presentation device 856, including presenting the determined sensory subtype, one or more therapies selected from the available SCS therapies, and one or more likelihoods of suitability each associated with a therapy of the selected one or more therapies for the determined sensory subtype.

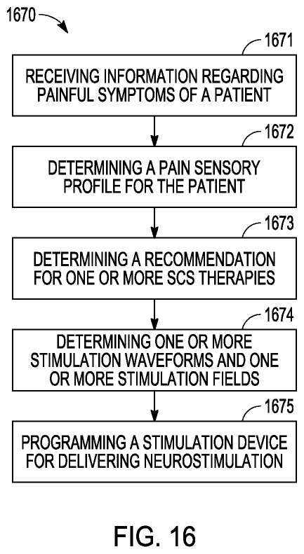

FIG. 16 illustrates an embodiment of a method 1670 for controlling delivery of neurostimulation from a stimulation device. Method 1670 can be performed using a neurostimulation system, such as any neurostimulation system discussed in this document (e.g., system 100, 500, or 600). A programming device, such as any programming device discussed in this document (e.g., programming device 102, programming device 302, external system 502, CP 630, RC 632, or external programming device 802), can be configured (e.g., programmed) to perform method 1670. In one embodiment, external storage device 818 includes a non-transitory computer-readable storage medium that includes instructions, which when executed by external programming device 802, cause external programming device 802 to perform method 1670. In various embodiments, method 1670 is performed for delivering neurostimulation from a stimulation device to a patient and controlling the delivery of the stimulation by a user.

At 1671, information regarding painful symptoms of the patient is received. This can include, for example, presenting a symptom questionnaire to the user and/or the patient, and receiving answers to the symptom questionnaire from the user and/or the patient. The symptom questionnaire can include, for example, a list of symptom types, measures of severity for each symptom type, and frequency for each symptom type (which relates the symptom type to one or more physical states of the patient).

At 1672, a pain sensory profile for the patient is determined using the received information. The pain sensory profile can be produced, for example, using the symptom questionnaire completed with the answers from the user and/or the patient. In various embodiments, the pain sensory profile can be determined for a specified body area of the patient. In various embodiments, the pain sensory profile can be determined for each body area of multiple specified body areas of the patient to result in a pain sensory map for the patient.

At 1673, a recommendation for one or more SCS therapies is determined using the determined pain sensory profile. In various embodiments, the recommendation for the one or more SCS therapies can be determined using the determined pain sensory map. In various embodiments, a sensory subtype is determined based on the pain sensory profile or pain sensory map, and the recommendation for the one or more SCS therapies is determined using the determined sensory subtype. In various embodiments, the sensory subtype is selected from known sensory subtypes, and the recommendation for the one or more SCS therapies is selected from available SCS therapies using a predetermined relationship mapping the known sensory subtypes to the available SCS therapies. In various embodiments, a likelihood of suitability for the sensory subtype associated with each therapy of the one or more SCS therapies is also determined for the recommendation of the one or more SCS therapies. The recommendation for the one or more SCS therapies can be presented with the determined sensory subtype, the one or more SCS therapies, and one or more likelihoods of suitability each associated with a therapy of the one or more SCS therapies.

At 1674, one or more stimulation waveforms and one or more stimulation fields are determined using the recommended one or more SCS therapies. In various embodiments, once the recommendation for the one or more SCS therapies is accepted by the user and/or the patient, the one or more stimulation waveforms and the one or more stimulation fields defining the one or more SCS therapies are determined in preparation for programming the stimulation device to deliver the one or more SCS therapies.

At 1675, the stimulation device is programmed for delivering neurostimulation to the patient and controlling the delivery of the neurostimulation according to the one or more stimulation waveforms and the one or more stimulation fields. Programming information representing or indicating the one or more stimulation waveforms and the one or more stimulation fields defining the one or more SCS therapies is transmitted to the stimulation device. In various embodiments in which the stimulation device includes an implantable stimulator, an external programmer is used to transmit the programming information to the implantable stimulator via a wireless communication link.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for delivering neurostimulation from a stimulation device to a patient, the system comprising:
a programming control circuit configured to program the stimulation device for controlling delivery of the neurostimulation according to one or more stimulation waveforms and one or more stimulation fields;
a sensory profiling circuit configured to receive information regarding painful symptoms of the patient and to determine a pain sensory profile for the patient using the received information; and
a stimulation control circuit configured to determine a recommendation for one or more spinal cord stimulation (SCS) therapies using the determined pain sensory profile and to determine the one or more stimulation waveforms and the one or more stimulation fields using the recommended one or more SCS therapies.

2. The system of claim 1, comprising a user interface including a presentation device, a user input device, and an interface control circuit including the sensory profiling circuit and the stimulation circuit, wherein the sensory profiling circuit is configured to receive the information regarding painful symptoms of the patient using the user input device, and the stimulation control circuit is configured to present the recommendation using the presentation device.

3. The system of claim 2, wherein the sensory profiling circuit is configured to present a symptom questionnaire using the presentation device, to receive answers to the symptom questionnaire using the user input device, and to produce the pain sensory profile using the received answers, the symptom questionnaire including a list of symptom types, a measure of severity for each symptom type of the list of symptom types, and a frequency for each symptom type of the list of symptom types, the frequency associating the each symptom type with each physical state of a list of physical states of the patient.

4. The system of claim 3, wherein the sensory profiling circuit is configured to produce the pain sensory profile for each body area of multiple specified body areas of the patient to result in a pain sensory map for the patient, and the stimulation control circuit is configured to determine the recommendation for the one or more SCS therapies using the resulted pain sensory map.

5. The system of claim 4, wherein the stimulation control circuit is configured to determine a sensory subtype based on the pain sensory map, and the stimulation control circuit is configured to determine the recommendation for the one or more SCS therapies using the determined sensory subtype.

6. The system of claim 5, wherein the stimulation control circuit is configured to receive one or more additional factors and to determine the recommendation for the one or more SCS therapies using the determined sensory subtype and the received one or more additional factors, the one or more additional factors including at least one of a stimulation-related factor, a disease-related factor, a demographic factor, or a behavioral factor.

7. The system of claim 5, wherein the stimulation control circuit is configured to determine the recommendation for the one or more SCS therapies using a predetermined relationship mapping known sensory subtypes to available SCS therapies.

8. The system of claim 5, wherein the stimulation control circuit is further configured to determine a likelihood of suitability for the sensory subtype associated with each therapy of the one or more SCS therapies and to determine the one or more stimulation waveforms and the one or more stimulation fields based on the recommended one or more SCS therapies and the associated one or more likelihoods of suitability.

9. The system of claim 8, wherein the stimulation control circuit is further configured to determine the likelihood of suitability for the sensory subtype associated with each therapy of the one or more SCS therapies using a predetermined lookup table providing the likelihood of suitability for each sensory subtype of the known sensory subtypes associated with each therapy of the available SCS therapies.

10. The system of claim 8, wherein the stimulation control circuit is configured to present the recommendation showing the determined sensory subtype, one or more therapies selected from the available SCS therapies, and one or more likelihoods of suitability each associated with a therapy of the selected one or more therapies for the determined sensory subtype.

11. A method for delivering neurostimulation from a stimulation device to a patient, the method comprising:
receiving information regarding painful symptoms of the patient;
determining a pain sensory profile for the patient using the received information using a processor of a programming device;
determining a recommendation for one or more spinal cord stimulation (SCS) therapies using the determined pain sensory profile using the processor;
determining one or more stimulation waveforms and one or more stimulation fields using the recommended one or more SCS therapies using the processor; and
programming the stimulation device, using the programming device, for controlling delivery of the neurostimulation according to the one or more stimulation waveforms and the one or more stimulation fields.

12. The method of claim 11, wherein receiving the information regarding painful symptoms of the patient comprises:
presenting a symptom questionnaire using a presentation device;
receiving answers to the symptom questionnaire using a user input device; and
producing the pain sensory profile using the received answers using the processor.

13. The method of claim 12, wherein presenting the symptom questionnaire comprising:
presenting a list of symptom types;
presenting measures of severity each associated with a symptom type of the list of symptom types; and
presenting frequencies each associated with a symptom type of the list of symptom types and relating the each associated symptom type to one or more physical states of the patient.

14. The method of claim 11, wherein determining the pain sensory profile for the patient comprises determining the pain sensory profile for a specified body area of the patient.

15. The method of claim 14, wherein determining the pain sensory profile for the patient comprises determining the pain sensory profile for each body area of multiple specified body areas of the patient to result in a pain sensory map for the patient, and determining the recommendation for one or more SCS therapies using the determined pain sensory profile comprises determining the recommendation for one or more SCS therapies using the determined pain sensory map.

16. The method of claim 15, wherein determining the recommendation for one or more SCS therapies using the determined pain sensory profile comprises:
determining a sensory subtype based on the pain sensory map:
determining the recommendation for the one or more SCS therapies using the determined sensory subtype.

17. The method of claim 16, wherein determining the sensory subtype comprises selecting the sensory subtype from known sensory subtypes, and determining the recommendation for the one or more SCS therapies comprises selecting the one or more SCS therapies from available SCS therapies using a predetermined relationship mapping the known sensory subtypes to the available SCS therapies.

18. The method of claim 17, wherein determining the recommendation for the one or more SCS therapies using the determined sensory subtype further comprises determining a likelihood of suitability for the sensory subtype associated with each therapy of the one or more SCS therapies.

19. The method of claim 18, further comprising presenting the recommendation for the one or more SCS therapies, including presenting the determined sensory subtype, the one or more SCS therapies, and one or more likelihoods of suitability each associated with a therapy of the one or more SCS therapies.

20. A non-transitory computer-readable storage medium including instructions, which when executed by a system, cause the system to perform a method for delivering neurostimulation from a stimulation device to a patient, the method comprising:
receiving information regarding painful symptoms of the patient;
determining a pain sensory profile for the patient using the received information;
determining a recommendation for one or more spinal cord stimulation (SCS) therapies using the determined pain sensory profile;
determining one or more stimulation waveforms and one or more stimulation fields using the recommended one or more SCS therapies; and
programming the stimulation device for controlling delivery of the neurostimulation according to one or more stimulation waveforms and one or more stimulation fields.
